(19) United States
(12) Patent Application Publication
(10) Pub. No.: US 2020/0390835 A1
(43) Pub. Date: Dec. 17, 2020

(54) Composition and a method for inhibiting the growth of pathogens causing bovine liver abscesses

(71) Applicants: Jacques Mathieu, Houston, TX (US); Pedro Alvarez, Houston, TX (US)

(72) Inventors: Jacques Mathieu, Houston, TX (US); Pedro Alvarez, Houston, TX (US)

(21) Appl. No.: 16/946,249

(22) Filed: Jun. 12, 2020

Related U.S. Application Data
(60) Provisional application No. 62/860,983, filed on Jun. 13, 2019.

(51) Int. Cl. A61K 35/76; A61P 31/04; A61K 9/00

(52) U.S. Cl. CPC A61K 35/76; A61K 9/0053; A61P 31/04

(57) ABSTRACT

The present invention relates to a composition and method for inhibiting the growth of pathogens causing liver abscesses in bovines. The composition comprises of one or more broad host-range (polyvalent) bacteriophages capable of infecting and lysing or more Fusobacterium species and an acceptable carrier for delivering said composition to said bovine. The bacteriophages are isolated and characterized for infecting and inhibiting the growth of pathogens. Particularly, the composition is orally administered daily to bovines. Moreover, the present invention relates to the method for controlling and inhibiting the growth of pathogens causing liver abscesses in bovines comprising sampling of cattle rumen; preparation of phage pools; isolation of polyvalent F. necrophorum phages; quantification of Fusobacterium species level in cattle rumen; creation of a Fusobacterium strain library; and genome sequencing of Fusobacterium species.

### TECHNICAL FIELD

The present invention generally relates to bacteriophages for pathogen control in livestock. More specifically, the present invention discloses development of polyvalent bacteriophages as an alternative to the use of antibiotic feed additives in livestock for the control of the growth of pathogens causing liver abscesses in bovines.

### BACKGROUND OF THE INVENTION

The beef and cattle industry has witnessed major changes over the past several decades with respect to feeding practices. In order to improve beef production efficiency, effectively utilize local agricultural byproducts, and meet consumer demand, feedlots were established that utilize high-energy, grain-based rations to maximize cattle growth. However, the feeding of grains to cattle can result in a transient drop in ruminal pH that causes detrimental changes to the rumen microbial community. One such change is an increase in the abundance of the bacterial genus Fusobacterium. Several different species of Fusobacterium are natural residents of the bovine rumen, including Fusobacterium varium, Fusobacterium necrophorum, Fusobacterium russi, Fusobacterium naviforme, Fusobacterium symbiosum, Fusobacterium plauti, Fusobacterium aquatile, Fusobacterium mortiferum, and Fusobacterium necrogenes.

Within the rumen microbial community, Fusobacterium necrophorum has been identified as the causative agent of liver abscesses, which is an economically important disease of the cattle industry. Fusobacterium necrophorum is a Gram(-), aerotolerant anaerobic bacteria that is commonly isolated from abdominal abscesses and respiratory tract infections in animals, and is the primary etiologic agent of liver abscesses in cattle. In cattle liver abscesses, Fusobacterium necrophorum may act synergistically with Trueperella pyogenes, Salmonella enterica, and other bacteria, which are considered secondary etiologic agents.

To reduce the ruminal abundance of Fusobacterium necrophorum in grain-fed cattle, the antibiotics tylosin and virginiamycin are often provided as a component of the cattle feed. The use of these antibiotics in cattle feed is reported to reduce the occurrence of liver abscesses by approximately 70% in feedlot cattle. However, the use of in-feed antibiotics may negatively affect the growth of beneficial and commensal bacterial species while promoting the growth of bacteria harboring antibiotic resistance genes. Thus, alternatives to antibiotics are desirable to maintain healthy rumen and gut microbiomes and to mitigate the proliferation of antibiotic resistant bacteria.

One such approach that can be utilized to overcome the issue of liver abscesses in bovines is the use of bacteriophages. A bacteriophage, or phage, is a virus that infects only bacterial cells. They are abundantly present in nature and can be isolated from sewage water, soil, feces, sediment, and other biological and environmental sources. Bacteriophage can attach to bacterial cells with their tail fibers and inject their genetic material into the bacterium. Phages are categorized based on the type of their life cycle, the lytic cycle cause lysis of the bacterium with the release of multiple phage particles whereas in lysogenic phase the phage DNA is incorporated into the bacterial genome. Lysogeny does not result in immediate lysis of the host. Lytic phages have several potential applications in the food industry as biocontrol agents, biopreservatives, and as tools for detecting pathogens. The use of bacteriophage has also been proposed as an alternative to antibiotics in maintaining animal health. Several experiments on poultry have been conducted with bacteriophage as feed additives instead of dietary antibiotics. Two unique features of phage relevant for food safety are that they are harmless to mammalian cells and have high host specificity, keeping the natural microbiota undisturbed. Numerous bacteriophages preparations have been approved for agricultural and food safety applications.

A number of solutions were introduced in order to solve the abovementioned problems. In one of the closest relevant arts, U.S. Pat. No. 7,207,289 B2 which discloses a method of increasing beef production in cattle with feed additives, comprising feeding cattle with feed, comprising an effective amount of an ionophore in combination with a macrolide antibiotic, and thereafter feeding cattle with feed, comprising zilpaterol and essentially no ionophore or macrolide antibiotic for the succeeding about 20 to 40 days. However, the art does not provide any alternative solution to the use of antibiotics as feed additives. Furthermore, the method mentioned above does not provide a suitable alternative to the use of antibiotics as feed additives, as bacteria develop resistance to the antibiotics over time.

Another relevant art EP0356192 A2 discloses pharmaceutical compositions for use in the prevention and treatment of liver abscesses in animals by combining a first component which is a member of the Virginiamycin family of antibiotics with a second component which is a member of the ionophore family of antibiotics and to the use of a member of the Virginiamycin family of antibiotics in the manufacture of a medicament for preventing and treating liver abscesses in animals. Although the relevant art disclosed above mentions the composition to treat and prevent liver abscesses, but still does not mention the alternative to the use of antibiotics as a feed additive.

Another relevant art U.S. Ser. No. 10/220,085 B2 discloses protecting against, treating, and detecting Fusobacteria infections. Compositions and methods derived from nucleic acid and protein sequences of a 40 kDa adhesin protein are provided to protect against, treat, and detect Fusobacteria infections in a subject. In one aspect, vaccines capable of inducing an immune response to a 40 kDa adhesin protein are used to protect against Fusobacteria infection. Also, nucleic acid molecules, proteins, immunogens, antibodies, and antisense molecules derived from the sequences of the 40 kDa adhesin protein may be used to protect against, treat, and detect Fusobacteria infections in a subject. Although the relevant art disclosed above mentions the composition and method for the treatment against Fusobacteria infections but still does not mention the use of bacteriophages as a feed additive for inhibiting the growth of pathogens causing liver abscesses.

Another relevant art US20020114817 A1 discloses a method of producing a vaccine for the prevention of F. necrophorum bacterial infections, comprising isolating the F. necrophorum bacteria from a bovine species, growing the bacteria in a suitable growth medium for a period equal to between about 10 hours and about 18 hours so as to achieve a bacterial population equal to at least 1×10⁵ CFU/ml, terminating the growth, and using a whole cell culture to form the vaccine. Additionally, the present invention relates to the vaccine comprised of a killed whole cell population of the F. necrophorum bacteria taken from a bovine. The present invention further relates to a method for preventing footrot and liver abscesses caused by F. necrophorum bacteria. Although the relevant art disclosed above mentions the method of producing a vaccine for the prevention of F. necrophorum bacterial infections but still does not mention the use of bacteriophages as a feed additive for inhibiting the growth of pathogens causing liver abscesses.

The conventional method of inhibiting the growth of Fusobacterium necrophorum by the use of antibiotics has a drawback of selecting microbial strains resistant to the antibiotics, thereby degrading the effect of antibiotics and hence, increasing the proliferation of antibiotic-resistant bacteria.

The present invention solves the problem of antibiotic resistance of microbial strains by utilizing bacteriophage as an alternative to the use of antibiotics as a feed additive, thereby inhibiting the growth of pathogens causing liver abscesses in bovine.

All the problems, disadvantages and the limitations of the above mentioned relevant and conventional arts being overcome by the method of the present invention which has various technical advancements and certainly economic benefits over the conventional arts.

### SUMMARY OF THE INVENTION

The present invention discloses a safe, natural, and environmental-friendly method for controlling the growth of pathogens causing liver abscesses, such as Fusobacterium necrophorum, in cattle rumen and gut microbiomes. More specifically, in one embodiment, the present invention is a composition for inhibiting the growth of pathogens causing liver abscesses in bovines comprising one or more bacteriophages capable of infecting and lysing one or more Fusobacterium species; and an acceptable carrier for delivering said composition to said bovine. In another embodiment the composition of one or more bacteriophages is provided in a stabilized form as a feed additive, and that are capable of lysing Fusobacterium species in cattle rumen or hindgut regions once ingested. In an alternative embodiment, the invention is a composition of one or more bacteriophages that are capable of lysing secondary etiological agents associated with liver abscesses, including, but not limited to, Trueperella pyogenes and Salmonella enterica. Said secondary etiological agents may directly or indirectly influence the abundance of Fusobacterium species and affect its ability to form liver abscesses in cattle.

Bacteriophages present in said compositions may consist of narrow host range bacteriophages specific to one or more strains of Fusobacterium species. Alternatively, bacteriophages present in said compositions may consist of broad host-range (polyvalent) bacteriophages capable of infecting more than one species of bacteria that are directly or indirectly associated with liver abscess occurrence. The present inventors have found that broad host-range bacteriophages may achieve higher titers and better microbial control when such bacteriophages are able to infect multiple hosts within a single microbiome, and that bacteriophage compositions developed using broad host-range bacteriophages may out-perform bacteriophage compositions developed using narrow host-range bacteriophages.

In one of the preferred embodiments of the present invention, the bacteriophages are in combination (cocktail) or a single phage species. Moreover, the invention develops a phage cocktail for the active treatment of Fusobacterium species in cattle rumen. The composition comprising one or more bacteriophages is delivered to the bovine by an acceptable carrier which comprises a material selected from the group of water, a buffered solution, or feed. In yet another embodiment, the composition is orally administered daily or weekly or monthly to said bovine. The composition is orally administered as a single dose or multiple doses to said bovine.

In one of the preferred embodiments, the present invention relates to a method for controlling and inhibiting the growth of pathogens causing liver abscesses in bovine comprising sampling of cattle rumen; preparation of phage pools; isolation of polyvalent F. necrophorum phages; quantification of Fusobacterium species level in cattle rumen; creation of a Fusobacterium strain library; and genome sequencing of Fusobacterium species. For the purpose of sampling, samples of cattle rumen are selected from unfiltered rumen fluid samples from freshly harvested cattle originating from five different feedlots. Furthermore, said bacteriophage pools are selected from the group of environmental sources consisting of soil, fecal, rumen fluid, activated sludge, and water samples. Preparation of bacteriophage pools comprises addition of elution fluids to samples. Furthermore, the elution fluids comprise 10 mM sodium pyrophosphate, 250 mM glycine (pH 8), SM buffer, or phosphate-buffered saline (pH 7.2). For solids, 1.0 g per 3-5 mL eluent. For slurries, an equal volume of eluent is added to each sample (e.g., 3 mL to 3 mL).

In another embodiment of the present invention, new F. necrophorum phages via prophage induction are isolated. Quantification of Fusobacterium species level in cattle rumen comprises of Fusobacterium varium in abundance which is equal to at least 2×10⁶ cells/mL. The abundance of Fusobacterium species is increased by treatment with enrichment cultures in selective media containing 50 μM lactate. Selective isolation of Fusobacterium from rumen fluid samples is used to create a Fusobacterium strain library.

In one of the preferred embodiment of invention, the present invention discloses bacteriophage as an alternative to the use of antibiotics as a feed additive, thereby inhibiting the growth of Fusobacterium species in bovines.

### BRIEF DESCRIPTION OF DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the method for the isolation of broad host-range bacteriophages.

FIG. 2 shows the change in concentration of Salmonella enterica following treatment with the narrow host-range phage PSEM-1.

FIG. 3 shows the reduction in Salmonella enterica following treatment with the broad host-range phage PSEP-1.

FIG. 4 shows a pie chart illustrating 16S metagenomic sequence analysis.

FIG. 5 shows strategies for assessing phage activity within phage pools.

FIG. 6 illustrates Fusobacterium plaque assays.

### DETAILED DESCRIPTION OF THE INVENTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The present invention relates to polyvalent bacteriophages as livestock feed additives and also as an alternative to antibiotics for the control and inhibition of pathogens causing bovine liver abscesses. The bacteriophages added in the livestock feed as an alternative to antibiotics inhibit the growth of pathogens causing liver abscesses such as Fusobacterium necrophorum, Salmonella enterica, etc. Moreover, the present invention provides an effective composition of one or more bacteriophage which is capable of infecting and inhibiting the growth of Fusobacterium species. The composition can be orally administered to the animal. Furthermore, the composition can be incorporated in the livestock feed as an additive to reduce the growth of Fusobacterium species in the rumen of the cattle.

The preferred embodiment of the present invention is to develop a phage cocktail for the active treatment of Fusobacterium species in cattle rumen. The method of the present invention comprises rumen sampling, processing, and community analysis. In order to isolate bacteriophages active against Fusobacterium species, samples were obtained from cattle rumen, which is a natural habitat for these bacteria. For this purpose, 50 unfiltered rumen fluid samples from freshly harvested cattle originating from five different feedlots were acquired. Various methods for preparing bacteriophage pools are utilized and smaller volumes are used for screening to identify the most promising strategies. This includes combinations of filtration, centrifugation, precipitation, and enrichment. Several techniques are utilized to determine the efficiency of each method by quantifying phage-like particles. These include UV spectroscopy, fluorescence microscopy after SYBR Gold staining, flow cytometry after SYBR Gold staining, electron microscopy, and plaque assays on common, permissive hosts. Interestingly, it is found that phage titers in filtered samples are lower than those typically found in other environmental samples. Titers are somewhat higher when using 0.45 μm filters rather than 0.22 μm filters, suggesting the presence of some larger phages. Additionally, titers appeared to be reduced when using chloroform to eliminate contaminating bacteria, suggesting some phages may possess lipids as part of their structure.

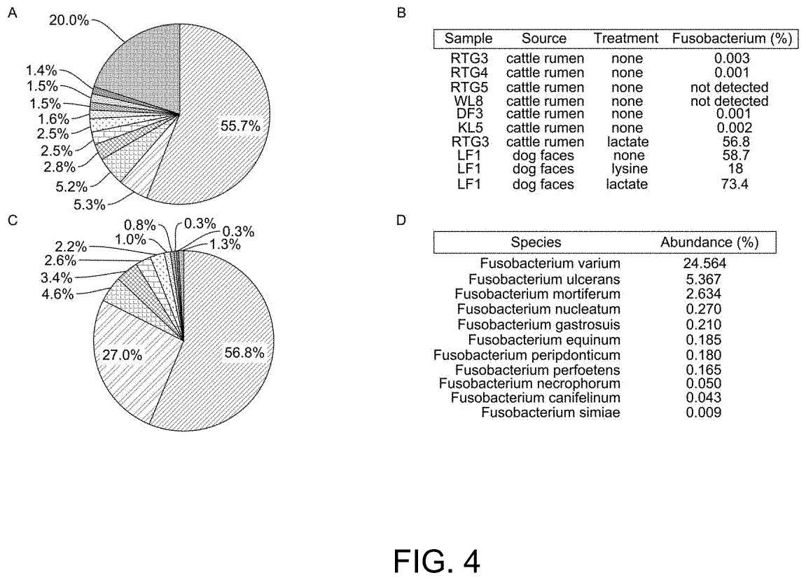

FIG. 4 illustrates microbial community analysis via 16S rRNA gene sequencing conducted on several rumen samples to determine the presence and relative abundance of Fusobacterium species. FIG. 4A illustrates top ten genera detected in rumen fluid sample RTG3 which are Prevotella, Treponema, Ruminococcus, Fibrobacter, Bacteroides, Hungateiclostridiaceae, Selenomonas, Roseburia, Lachnoclostridium, Oscillibacter and others. FIG. 4B illustrates characterization of rumen fluid microbial communities is consistent with previous studies, and demonstrates a relatively low abundance of Fusobacterium in cattle rumen 0.003%). Enrichment of RTG3 in selective media containing 50 μM lactate greatly increases the abundance of Fusobacterium to 56.8%. Dog fecal samples are also assessed for Fusobacterium and are found to contain a high abundance (>40% in all samples). Enrichment with lactate similarly increased abundance to 73.4%. FIG. 4C illustrates top ten genera detected in enrichments prepared with RTG3 and lactate. Besides Fusobacterium, the enrichments are selected for the growth of Clostridium and Proteobacteria. FIG. 4D illustrates total relative abundances of Fusobacterium species which states that the percentage of abundance of F. varium is the highest, having 24.564% abundance. The species-level analysis using full-length 16S rRNA primers and nanopore sequencing reveals the presence of numerous Fusobacterium species in the RTG3/lactate enrichment. Notably, F. varium dominates in all samples assessed, with F. necrophorum typically detected at concentrations 2- to 4-orders of magnitude lower.

Accordingly, in another embodiment, phage isolation, characterization and sequencing is conducted. The experiments result in the isolation of nine new phages capable of infecting F. necrophorum, and another 24 phages capable of infecting F. varium. Importantly, it is demonstrated that all of the isolated F. necrophorum phages are polyvalent (capable of infecting different species within the Fusobacterium genus). Moreover, phage pools from 50 rumen samples are prepared, Fusobacterium phages are not obtained when directly screening these pools using spot and plaque assays. Phages are isolated against other bacterial species from these pools, and it is verified that they contain sufficient numbers of viral particles. It is suspected that this is due to the low abundance of Fusobacterium within cattle rumen, as demonstrated by 16S metagenomic analyses.

To circumvent this, Fusobacterium enrichment is conducted using lactate and phages pools are prepared from these cultures. Phage pools are prepared and tested from a wide variety of different environmental sources, including sewage water, activated sludge, sea water, pond water, soil, and sediment, but still F. necrophorum phages are not obtained after many attempts.

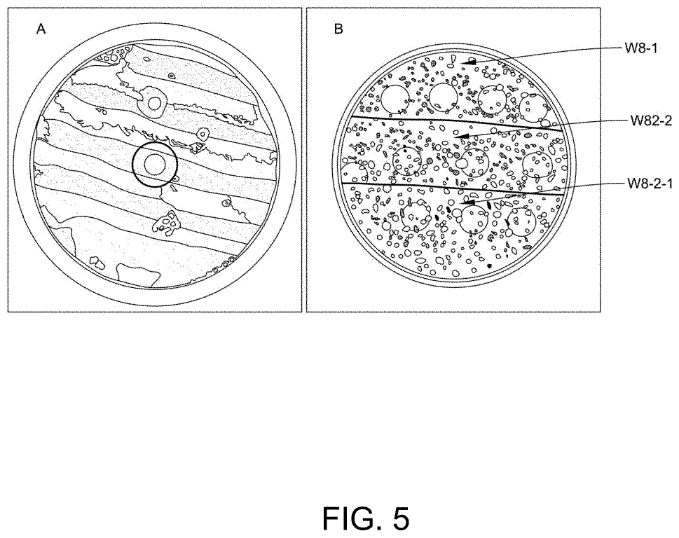

FIG. 5 illustrates the strategies for assessing phage activity within phage pools. FIG. 5A illustrates streak assay with Fusobacterium growing on the surface of BHI (Brain Heart Infusion) blood agar. Phage pools are spotted along the streaked areas. Black circle indicates spot displaying growth inhibition. Further, FIG. 5B illustrates spot assay using a reduced blood concentration in the bottom agar (1%) and normal blood concentration in the top agar (5%). Glycerol is added in order to enhance the transparency which further improves spot visualization. Furthermore, it is possible that early attempts to isolate phages are hindered by the difficulty of growing F. necrophorum in double layer agar assays. However, it is found that obtaining confluent growth in such assays requires strict adherence to the conditions identified, which are strain-dependent and highly impacted by the initial inoculum concentration and growth state. It is also considered that this might be due to the particular strains used as the isolation hosts, so additional F. necrophorum strains are obtained and re-tested the previously prepared phages pools using these new hosts and other Fusobacterium species. This approach proves successful for obtaining phages active against F. varium 8501 using phage pools prepared from lactate-supplemented rumen fluid enrichment cultures. Subsequent rescreening of phage pools prepared from other lactate-enriched cultures yielded a wide range of phages that displayed activity towards F. varium, but not F. necrophorum. Twenty-four of these phages (all from different rumen samples) are purified, amplified, and archived for future characterization.

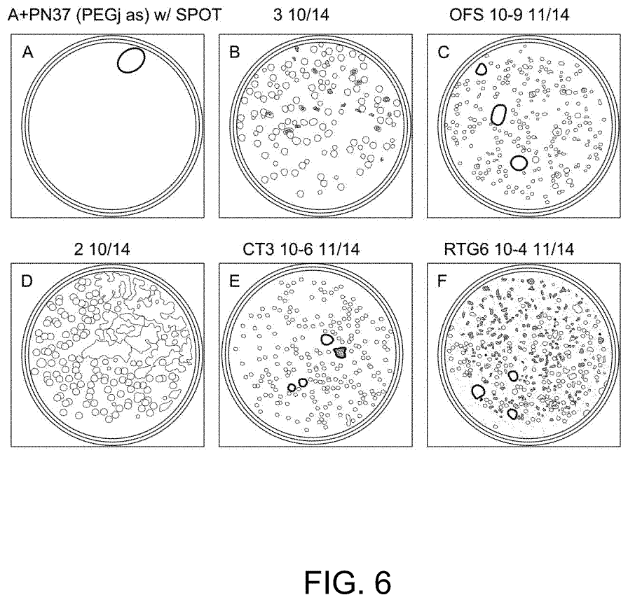

FIG. 6 illustrates Fusobacterium plaque assays. For the purpose of Fusobacterium plaque assay a modified plaque assay is developed to accommodate the growth requirements of Fusobacterium while still allowing for plaque visualization. FIG. 6A illustrates phage φFN37 with F. necrophorum strain A as host; FIG. 6B illustrates phage φWL8-3 with F. varium 8501 as host; FIG. 6C illustrates ϕDF3-1 with F. varium 8501 as host; FIG. 6D illustrates ϕWL8-2 with F. varium 8501 as host; FIG. 6E illustrates ϕCT3-1 with F. varium 8501 as host; and FIG. 6F illustrates ϕFNR6 with F. necrophorum RTG3 as host.

The ease with which F. varium phages are isolated is due to their dominance within the samples used herein. Attempts to enrich for F. necrophorum routinely enriched for the faster-growing F. varium instead. However, directly streaking rumen samples onto selective media resulted in the isolation of F. necrophorum from approximately 50% of the samples screened. Based on this information, investigation of other sources of Fusobacterium, and attempts to induce prophages from the numerous F. necrophorum strains is done. FIG. 6A illustrates that the latter approach quickly yields a phage (ϕFN37) that infects and lyses two of the indicator strains (F. necrophorum strains A and ATCC 25286) as determined by both spot and plaque assays. In order to increase the number and diversity of phages recovered by prophage induction, selective media is utilized to isolate additional Fusobacterium strains from preserved rumen samples, bringing the library to twenty-one strains. From these, a total of nine F. necrophorum phages are isolated. Further analysis determined ϕFN37 is capable of lysing five of the twenty-one strains tested.

All nine F. necrophorum and four F. varium phages are subjected to genome sequencing using an Oxford Nanopore MinION. Single contig assemblies are obtained for two of the F. necrophorum phages and all of the F. varium phages. Multiple contigs are obtained for the remaining F. necrophorum phages, which are sufficient to conduct preliminary analyses, but not assemble complete genomes. All but one of the sequenced F. varium 8501 phages (ϕFF10-3) are predicted to be temperate based on the presence of prophage-related genes (e.g., integrases).

Furthermore, BLAST queries of the genome sequences have high similarity to previously deposited Fusobacterium chromosome or plasmid sequences. Genome sizes ranged from 39 kbp to 85 kbp for the completely assembled genomes. No genes encoding toxins or antibiotic resistance genes are detected, with the exception of ϕFF38, which encodes a homolog to virulence-associated protein E. As the GC content of Fusobacterium species is very low (˜30%), they inevitably contain larger, more numerous homopolymer tracts within their genomes. It is found that a large number of insertions are erroneously incorporated at these sites, resulting in frameshifts and more ORFs being identified than would be expected. Manual annotation is possible but extremely laborious. Illumina sequencing data, while highly accurate, often produces incorrect assemblies. However, preliminary tests using Illumina sequencing data in combination with Nanopore data indicated that hybrid assembly methods can produce high-quality, correctly assembled genomes. Furthermore, advances in genome sequencing have made it a relatively simple task to rapidly screen phages for unwanted genes and eliminate one of the major concerns associated with lysogeny. Accordingly, temperate phages are being increasingly investigated as biocontrol agents due to the difficulty isolating purely lytic phages for many anaerobic bacteria.

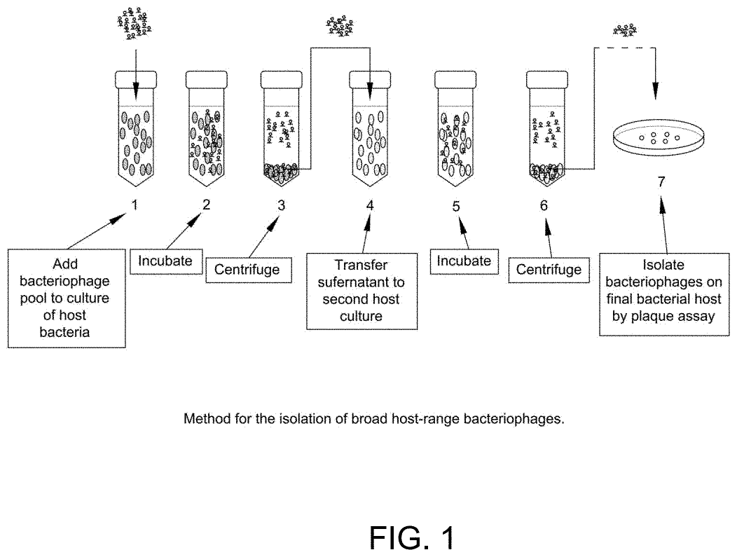

FIG. 1 illustrates the method for specific isolation of broad host range bacteriophages. The method involves the steps of adding bacteriophage pool to culture of host bacteria, followed by incubation and centrifugation. Further, the steps involve transferring of supernatant to second host culture, followed by incubation and centrifugation. Lastly, the method involves isolation of bacteriophages on final bacterial host by plaque assay. The bacteriophages added in the said composition can be of a narrow or broad host range. The most preferred bacteriophage is a broad host range bacteriophage or more commonly known as polyvalent bacteriophage. The favorable aspect of the bacteriophage is their host density dependency, which means that the bacteriophage is totally dependent on the density of the host. On the other hand, antibiotics and other pharmaceutical products are not dependent on the density of the host, making them unfavorable. The density of bacteria that must be present for bacteriophages to sustainably replicate is known as the proliferation threshold. Proliferation thresholds are dependent on several factors, including phage burst size, adsorption constants, and decay rates, but generally range from 10⁴ to 10⁶ cells/mL. Passive biocontrol describes cases where hosts are present below the proliferation threshold, and most target bacteria are removed by lysis following primary infection. During active biocontrol, host concentrations exceed the proliferation threshold and lysis-released bacteriophage is the dominant cause of removal. Generally, passive biocontrol is most dependent on the initial bacteriophage dose while active biocontrol relies on high host densities to achieve sustainable bacteriophage replication. As the temporal dynamics of bacterial densities in most microbiomes is not typically known prior to the use of bacteriophage-mediated biocontrol, passive treatment is suggested to be the most conservative approach, using inundative doses greater than 10⁸ phages/mL. However, such doses may be impractical when economic considerations are a factor. Thus, the use of broad host-range bacteriophages capable of infecting more than one bacterial species present in the rumen or hindgut is preferred.

While it is preferable to use naturally lytic bacteriophages, it is sometimes necessary to use naturally lysogenic bacteriophages. Preferably, deletion mutants of lysogenic bacteriophages are isolated after treatment with a chelating agent such as sodium pyrophosphate, EDTA, or sodium citrate. It is also preferable that isolated deletion mutants have lost their capacity to enter the lysogenic cycle. Such bacteriophages are isolated by using standard prophage induction protocols (e.g., incubation with mitomycin C) followed by incubation with a chelating agent at a concentration between 5-200 mM. Virulent deletion mutants are obtained by selection of those forming clear plaques on lawns of a suitable host.

Elution fluids are added to each sample. Preferred eluents include 10 mM sodium pyrophosphate, 250 mM glycine (pH 8), SM buffer, or phosphate-buffered saline (pH 7.2). For solids, 1.0 g is suggested per 3-5 mL eluent. For slurries, an equal volume of eluent is added to each sample (e.g., 3 mL to 3 mL). Solid and slurry samples are then shaken gently for 30 mins at room temperature. Some samples may be sonicated at low power for 1-3 mins to detach bacteriophages from particles. All samples are then centrifuged and subjected to filtration.

Depending on the sample source, samples may be prefiltered using 1-2.5 μm paper filter and a filter column apparatus with a vacuum pump. All samples are then filtered into sterile tubes or vials using 0.45 μm PVDF syringe filter (Durapore) and split equally among three vials. The present inventors have found that many broad host-range bacteriophages have larger genomes, and it is also known that a substantial percentage of rumen bacteriophages contain lipids. Thus, in order to limit the loss of large, lipid-containing bacteriophages (>220 nm), it is recommended that one aliquot of each 0.45 μm-filtered sample be retained without further treatment. A second aliquot is amended with 10% (v/v) chloroform to kill any bacteria that passed through the 0.45 μm PVDF or PES filter. The third aliquot is passed through a 0.22 μm PVDF or PES filter. It is suggested that all samples are stored at 4° C. for short-term use, or amended with an equal volume of a 50% glycerol solution and stored at −20° C. for long-term storage. Storage in capped 20 mL serum bottles is preferred as it allows for the removal of oxygen by sparging.

Spot tests are performed with each bacteriophage pool to determine the presence of lytic bacteriophages. Various Fusobacterium species strains are streaked onto the surface of BHI blood agar plates and allowed to dry for 5 minutes. Then, 5-10 μL of each phage pool is spotted onto the streaked regions. Plates are allowed to dry for several minutes and then transferred to anaerobic conditions and maintained at 37° C. until growth is visible. Cleared regions within the bacterial streaks are assumed to be due to the presence of lytic bacteriophages. Pools that test positive during spot tests are further subjected to plaque assays for bacteriophage isolation. For this method, one approach is that 200 μL samples of overnight cultures of each bacterial strain are added to 0.5% molten agar containing 5% defibrinated horse blood and kept at 48° C. in a dry bath. 100 μL of each bacteriophage pool is then added and the solution poured onto a 1.5% BHI agar plate. After the molten agar solidifies, each plate is transferred to anaerobic conditions and maintained at 37° C. until growth is observed. Plaques appearing within the bacterial lawn were purified by streaking three times, and the purified bacteriophages are amplified by incubation on plates containing their host, filtered, and stored at 4° C. until further use.

To isolate bacteriophages capable of infecting two or more bacterial species, a sequential, multi-host isolation method has been developed and used. (FIG. 1). This system is based on various optimality models that have enabled the prediction of factors favorable for the selection of polyvalent bacteriophages. Importantly, we have demonstrated that the isolation of bacteriophages capable of inter-order infectivity can be routinely achieved with the use of different sequential hosts, and the isolated bacteriophages can have similar efficiencies on all the isolation hosts. This corroborates the emerging perception that polyvalent bacteriophages are more widespread than previously perceived, and has enabled the development of more refined techniques, allowing the routine isolation of polyvalent bacteriophages.

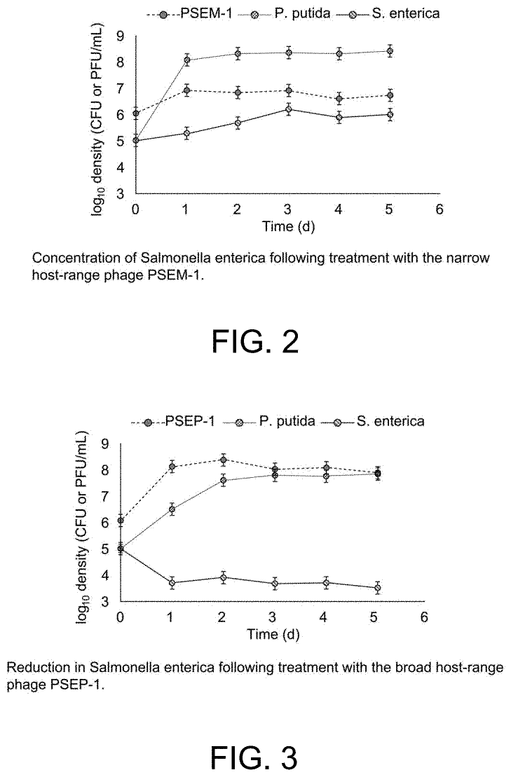

In another embodiment, the invention contains one or more bacteriophages capable of infecting more than one species of Fusobacterium. Alternatively, the invention contains one or more bacteriophages capable of infecting Fusobacterium necrophorum as well as a bacterium from a different genus. FIGS. 2 and 3 illustrates that narrow host-range bacteriophages are less effective than broad host-range phages in mixed bacterial system, particularly when the broad host-range bacteriophage can utilize multiple hosts within the system. When broad host-range bacteriophages are used, it is preferable to select bacteriophages that are more efficient at lysing the target host (e.g., Fusobacterium necrophorum) than the secondary hosts included to enhance proliferation.

Bacteriophage cocktails are typically used for microbial control instead of single phages to increase the breadth of host coverage, and reduce the development of resistance in the target bacteria. Preferably, bacteriophage cocktails are prepared in a rational manner, as a number of studies have shown that phages can interact with one another during co-infection. The interaction between two phages can have synergistic or antagonistic effects on a bacterial host. Therefore, phage compatibility is assessed using Appelmans' method by challenging cultures of host strains with bacteriophage pairs. Antagonistic and synergistic effects between bacterial species should also be considered when choosing phages with different host ranges.

Cattle rumen average 175 L, suggesting a dose must be 1.75×10¹³ bacteriophages for a passive treatment strategy, and likely greater when factoring in phage decay rates in the rumen. Optimization of phage production using high density fermentation might yield 10¹⁴ phages/L, however downstream processing and storage will significantly reduce phage viability. Furthermore, multiple phages are generally mixed together in a single cocktail to ensure broader host coverage and a reduced chance for resistance development. Therefore, a single dose using a passive treatment strategy can easily cost hundreds of dollars; a number that well exceeds the typical profit per head of cattle. While this is a simplified example, and economies of scale would likely further decrease this cost, it is important to consider these numbers in comparison to passive treatment. If phage dose could be lowered 1000-fold using active treatment, then a dose that previously cost $500.00 (developed with a passive treatment strategy in mind) would be $0.50 or less if active treatment could be used. Additionally, passive treatment often requires multiple doses, while single doses can be effective when using active treatment. The ability of polyvalent phages to target multiple species may facilitate the use of active treatment even when the target host is present below proliferation thresholds as potentially more abundant hosts can be used for replication. These secondary hosts can be indigenous species, or supplied as direct-fed microbials.

In the preferred implementation, bacteriophage cocktails are administered to cattle with feed or water in such a manner as to provide approximately 10¹¹ bacteriophages per dose. Doses may be administered at any interval or time, but it is recommended to begin administration simultaneous with the transition from a forage-based diet to concentrated feed. For example, a bacteriophage cocktail consisting of a lyophilized powder may be added to daily cattle rations using a micro machine, which is typically used in feedlots to add micro-ingredients such as antibiotics. Bacteriophage cocktails can also be added to water or provided directly by oral dosing. In order to facilitate storage and application, bacteriophage cocktails may be stabilized by encapsulation, lyophilization, or any other method that protects the bacteriophages from degradation and maintains viability.

### EXAMPLE

### Example 1. Efficacy of Polyvalent Versus Narrow Host-Range Bacteriophages in Controlling Salmonella enterica in a Two-Species Culture

Bacteriophage pools were prepared using filtered activated sludge from a wastewater treatment plant. Narrow host-range phages capable of lysing Salmonella enterica were isolated by plaque assay and purified using standard methods. Polyvalent bacteriophages capable of lysing both Pseudomonas putida and Salmonella enterica were isolated from activated sludge using a sequential, multi-host isolation method (FIG. 1). The host ranges of isolated bacteriophages were verified using spot tests on lawns of each host. Cultures were inoculated with 10⁵ CFU/mL of both Salmonella enterica and Pseudomonas putida, and amended with either a narrow host-range bacteriophage active against only Salmonella enterica (PSEM-1) or a polyvalent bacteriophage capable of infecting both Salmonella enterica and Pseudomonas putida (PSEP-1).

Continued growth of Salmonella enterica was observed over five days in cultures treated with PSEM-1 (FIG. 2), but not PSEP-1 (FIG. 3). Growth of Pseudomonas putida continued in both cultures. The efficiency of plating (EOP) of PSEP-1 was five-fold higher on Salmonella enterica than Pseudomonas putida. PSEP-1 titers were over 10-fold higher than PSEM-1, putatively due to its capacity to replicate in both hosts.

What is claimed is:

1. A composition for inhibiting the growth of pathogens causing liver abscesses in bovines comprising;
one or more bacteriophages capable of infecting and lysing one or more Fusobacterium species; and
an acceptable carrier for delivering said composition to said bovine.

2. The composition of claim 1, wherein the said bacteriophages are broad host-range (polyvalent).

3. The composition of claim 1, wherein the said bacteriophages are capable of infecting and lysing said pathogens.

4. The composition of claim 1, wherein the said bacteriophages are isolated and characterized for infecting and inhibiting the growth of said pathogens.

5. The composition of claim 1, wherein the said bacteriophages are isolated with a predetermined host-range based on rumen microbial composition.

6. The composition of claim 1, wherein the said bacteriophages are in combination (cocktail) or a single phage species.

7. The composition of claim 1, wherein the said acceptable carrier comprises a material selected from the group consisting of water, a buffered solution, or feed.

8. The composition of claim 1, wherein said composition is orally administered daily to said bovine.

9. The composition of claim 1, wherein said composition is orally administered weekly to said bovine.

10. The composition of claim 1, wherein said composition is orally administered monthly to said bovine.

11. The composition of claim 1, wherein said composition is orally administered as a single dose to said bovine.

12. The composition of claim 1, wherein said composition is orally administered in multiple doses to said bovine.

13. A method of pathogen control for inhibiting the growth of pathogens causing liver abscesses in bovine comprising:
sampling of cattle rumen;
preparation of phage pools;
isolation of polyvalent F. necrophorum phages;
quantification of Fusobacterium species level in cattle rumen;
creation of a Fusobacterium strain library; and
genome sequencing of Fusobacterium species.

14. The method of claim 13, wherein samples of cattle rumen are selected from unfiltered rumen fluid samples from freshly harvested cattle originating from different feedlots.

15. The method of claim 13, wherein the said bacteriophage pools are selected from the group of environmental sources consisting of soil, fecal, rumen fluid, activated sludge, and water samples.

16. The method of claim 13, wherein preparation of said bacteriophage pools comprises addition of elution fluids to said samples.

17. The method of claim 16, wherein said elution fluids comprising 10 mM sodium pyrophosphate, 250 mM glycine (pH 8), SM buffer, or phosphate-buffered saline (pH 7.2) for solids, 1.0 g per 3-5 mL eluent, for slurries, an equal volume of eluant added to each sample (e.g., 3 mL to 3 mL).

18. The method of claim 13, wherein F. necrophorum phages are isolated via prophage induction.

19. The method of claim 13, wherein said quantification of Fusobacterium species level in cattle rumen comprises of Fusobacterium varium in abundance which is equal to at least 2×10⁶ cells/mL.

20. The method of claim 13, wherein the abundance of Fusobacterium species is increased by treatment with enrichment cultures in selective media containing 50 μM lactate.

21. The method of claim 13, wherein selective isolation of Fusobacterium from rumen fluid samples is used to create said Fusobacterium strain library.
